(19) United States
(12) Patent Application Publication
(10) Pub. No.: US 2026/0224617 A1
(43) Pub. Date: Aug. 6, 2026

(54) COMPOSITIONS AND METHODS FOR TREATMENT OF SPACEFLIGHT-ASSOCIATED MITOCHONDRIAL DAMAGE

(71) Applicant: The Sallie Astor Burdine Breast Foundation, Baton Rouge, LA (US)

(72) Inventors: Brent M. Segal, Pembroke, MA (US); Catherine Baucom, Baton Rouge, LA (US); Xianpeng Jiang, Baton Rouge, LA (US)

(21) Appl. No.: 19/154,006

(22) PCT Filed: Sep. 8, 2023
(86) PCT No.: PCT/US23/32294

§ 371 Date: Aug. 6, 2025

Related U.S. Application Data
(60) Provisional application No. 63/444,741, filed on Feb. 10, 2023.

(51) Int. Cl. A61K 35/15; C12N 5/00

(52) U.S. Cl. CPC A61K 35/15; C12N 5/526; C12N 5/562

(57) ABSTRACT

Presented herein are compositions and methods involving mitochondrial organelle transplantation for use in the treatment and/or prevention and/or amelioration of spaceflight-associated mitochondrial damage (e.g., mitochondrial dysfunction).

### CROSS-REFERENCE TO PENDING APPLICATION

This application claims priority to and the benefit of U.S. Provisional Application No. 63/444,741, filed Feb. 10, 2023, the disclosure of which is incorporated by referenced herein in its entirety.

### FIELD

The subject matter described herein relates to compositions and methods for treatment of a subject. In certain embodiments, described herein are compositions and methods involving mitochondrial organelle transplantation for use in the treatment of spaceflight-associated (e.g., spaceflight-induced) mitochondrial damage (e.g., mitochondrial dysfunction) in a subject.

### BACKGROUND

It has been observed that the mitochondria of an astronaut may be negatively impacted by the low gravity, radiation, confined living spaces, and/or other conditions experienced on a space mission, for example, an extended mission on the International Space Station, or on missions to the Moon or Mars. Mitochondrial dysfunction may negatively impact the physiology and health of astronauts and animals during and/or after spaceflight.

In 2004, Nikawa et al. proposed that spaceflight-induced mitochondrial activity alteration was associated with muscle atrophy. The decreased mitochondrial activity was reported in the roots of space-flown soybean seedlings. The lower diffusion rates of solutes in microgravity during spaceflight reduced the hydrogen gradient at the mitochondrial intermembrane space, reducing electron transport chain and ATP synthesis. A recently comprehensive multi-omics analysis from 59 astronauts and hundreds of samples flown in space provides evidence of mitochondrial dysregulation as a central hub for space biology. Altered mitochondrial function, increased oxidative stress and DNA damage were found after analysis of urine and blood of astronauts. The oxidized mitochondrial DNA activated innate immune system inflammasome and induced inflammation. A prolonged exposure to space conditions induced the reduced mtDNA and mtRNA production and this phenomenon continued even after spaceflight.

Therefore, there exists a need for treatment of mitochondrial dysfunction in astronauts.

### SUMMARY

The present disclosure provided, among other things, compositions and methods involving mitochondrial organelle transplantation for use in the treatment and/or prevention and/or amelioration of spaceflight-associated mitochondrial damage (e.g., mitochondrial dysfunction). Described herein are methods of applying methods and compositions for the treatment of mitochondrial dysfunction suffered by an individual as a result of (or associated with) spaceflight, for example, an astronaut.

During long-term spaceflight, astronauts are subjected to a variety of environmental conditions, which can affect the behavior of mitochondria. For example, low gravity conditions (e.g., micro-gravity, zero gravity), cosmic rays (e.g., cosmic radiation) and radiation can all result in damage to mitochondria. Spaceflight may also result in or contribute to stress, sleep deprivation, and muscle weakness (e.g., due to low gravity conditions), among other conditions. Despite the care taken by aeronautical organizations to minimize these effects, for example, through radiation shielding and exercise, many astronauts will still be left with oxidative stress, inflammation, and other conditions as a result of their experiences in space.

Accordingly, the administration of autologous or allogeneic mitochondria to a subject who has experienced, is experiencing, or will experience spaceflight (e.g., long-term spaceflight) will help, among other things, restoration of mitochondrial function and alleviate oxidative stress and inflammation due to the environmental conditions experienced by astronauts during and after long-term spaceflight.

In one aspect, the invention is directed to a method for autologous or allogeneic transplantation of mitochondria in a subject (e.g., a human or animal subject) for treatment and/or prevention and/or amelioration of spaceflight-associated mitochondrial damage (e.g., mitochondrial dysfunction), said method comprising administering to said subject a composition (e.g., a pharmaceutical composition) comprising mitochondria isolated from the subject (e.g., for autologous transplantation) or isolated from a donor other than the subject (e.g., for allogeneic transplantation), wherein (i) the subject has engaged in spaceflight or is engaging in spaceflight at the time of administration of the composition and has or may have mitochondrial damage associated with said spaceflight, or (ii) wherein the subject is about to engage in spaceflight at the time of administration of the composition and said composition is administered to prevent or ameliorate spaceflight-associated mitochondrial damage.

In certain embodiments, the composition further comprises a mitochondrial storing buffer having a potassium ion concentration safe for administration to humans (e.g., wherein said mitochondrial storing buffer comprises a pharmaceutically acceptable carrier).

In certain embodiments, the administering step comprises parenterally administering (e.g., by intramuscular or intravenous injection) at least one-unit dose of said composition to said subject. In certain embodiments, the administering step comprises both intramuscular injection and intravenous injection of said composition to said subject.

In certain embodiments, the method further comprises (i) isolating said mitochondria from said donor other than the subject or (ii) isolating said mitochondria from said subject (e.g., at a time prior to the spaceflight).

In certain embodiments, isolating said donor mitochondria (or said pre-spaceflight-harvested subject mitochondria) comprises preparing cell lysate from tissue of the donor via tissue dissociation (e.g., using a bead-tube shake homogenizer).

In certain embodiments, isolating said donor mitochondria (or said pre-spaceflight-harvested subject mitochondria) comprises using a mitochondrial isolation buffer comprising a serine protease inhibitor (e.g., phenylmethylsulfonyl fluoride (PMFS)) (e.g., to prevent or reduce damage of donor mitochondria from digestive enzymes).

In certain embodiments, the method comprises isolating said donor mitochondria (or said pre-spaceflight-harvested subject mitochondria) without using an antibiotic.

In certain embodiments, the donor and the subject are not an HLA (human leukocyte antigen) match [e.g., not an identical match (e.g., based on match of 8 or 10 tested HLA markers) and/or not a haploidentical match (e.g., based on match of 8 or 10 tested HLA markers), and/or of indeterminate match status (e.g., no HLA markers tested prior to the administering step)].

In certain embodiments, the composition administered to the subject does not comprise an antibiotic (e.g., and wherein the subject is not administered an antibiotic).

In certain embodiments, the composition comprises mitochondria isolated from (i) human primary fibroblasts of the donor (or of the pre-spaceflight subject) and/or (ii) mesenchy mal stromal cells (MSCs) of the donor (or of the pre-spaceflight subject).

In certain embodiments, the method further comprises isolating the mitochondria from the donor (or of the pre-spaceflight subject), wherein the isolating step is conducted using a mitochondrial isolation buffer composition.

In certain embodiments, the mitochondrial isolation buffer composition comprises: a buffering agent; a chelating agent; a sugar; an agent that acts as a membrane stabilizer and/or oxygen radical scavenger and/or binder of Ca²⁺ and/or binder of free fatty acid; and a serine protease inhibitor.

In certain embodiments, the composition does not comprise an antibiotic.

In certain embodiments, the method further comprises storing the isolated mitochondria at a temperature below −40° C. (e.g., below −60° C., below −70° C., or below −80° C., e.g., using liquid nitrogen).

In certain embodiments, the method comprises administering to the subject an iron-chelating agent (e.g., desferrioxamine or deferasirox).

In certain embodiments, the method comprises administering to the subject an antioxidant and/or a probiotic.

In another aspect, the invention is directed to a mitochondrial isolation buffer composition (e.g., an aqueous solution) for use in performing the method of any one of claims 1 to 18 (e.g., for use in mitochondrial organelle transplantation), said composition comprising: a buffering agent [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an agent that acts as a membrane stabilizer and/or oxygen radical scavenger and/or binder of Ca²¹ and/or binder of free fatty acid (e.g., bovine serum albumin, BSA); and a serine protease inhibitor (e.g., phenylmethylsulfonyl fluoride (PMFS), also called phenylmethane sulfonyl fluoride) (e.g., wherein the composition further comprises isolated donor mitochondria, e.g., fibroblast mitochondria).

In certain embodiments, the composition does not comprise an antibiotic.

In another aspect, the invention is directed to a mitochondrial storing buffer composition (e.g., an aqueous solution) for use in performing the method of any one of claims 1 to 18 (e.g., for use in mitochondrial organelle transplantation), said composition comprising: one or more buffering agents [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)] [e.g., monopotassium phosphate (KH₂PO₄)]; a source of magnesium ion [e.g., magnesium chloride (MgCl₂)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an antioxidant [e.g., taurine]; a cytoprotective agent that binds to calcium ion [e.g., lactobionate or salt thereof, e.g., K-lactobionate]; and an agent that acts as a membrane stabilizer and/or oxygen radical scavenger and/or binder of Ca²⁺ and/or binder of free fatty acid (e.g., bovine serum albumin, BSA) (e.g., wherein the composition further comprises isolated donor mitochondria, e.g., fibroblast mitochondria).

In certain embodiments, the composition does not comprise an antibiotic.

In another aspect, the invention is directed to a kit comprising a donor mitochondria composition (e.g., an aqueous composition) in a unit dosage effective to treat and/or ameliorate, and/or prevent spaceflight-associated mitochondrial damage (e.g., mitochondrial dysfunction) in a subject, said donor mitochondria composition comprising: mitochondria isolated from tissue of a donor (e.g., the pre-spaceflight subject or a donor other than the subject) (e.g., fibroblast mitochondria); one or more buffering agents [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)] [e.g., monopotassium phosphate (KH₂PO₄)]; a source of magnesium ion [e.g., magnesium chloride (MgCl₂)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an antioxidant [e.g., taurine]; a cytoprotective agent that binds to calcium ion [e.g., lactobionate or salt thereof, e.g., K-lactobionate]; and an agent that acts as a membrane stabilizer and/or oxygen radical scavenger and/or binder of Ca²⁺ and/or binder of free fatty acid (e.g., bovine serum albumin, BSA).

In certain embodiments, the donor and the subject are not an HLA (human leukocyte antigen) match [e.g., not an identical match (e.g., based on match of 8 or 10 tested HLA markers) and/or not a haploidentical match (e.g., based on match of 8 or 10 tested HLA markers), and/or of indeterminate match status (e.g., no HLA markers tested prior to the administering step)].

In certain embodiments, the donor mitochondria composition does not comprise an antibiotic.

In certain embodiments, the kit further comprises instructions for optimizing the dose and/or frequency and/or route of administration of the composition.

In another aspect, the invention is directed to a method of treating spaceflight-associated mitochondrial damage (e.g., mitochondrial dysfunction), the method comprising: administering to a subject a composition comprising mitochondria, wherein the mitochondria is autologous or allogeneic mitochondria.

In certain embodiments, the subject has engaged in spaceflight prior to (e.g., at least an hour, at least a day, at least a week, at least a month, at least six months, at least a year, at least five years, or more) the administration of the composition.

In certain embodiments, the subject is engaging in spaceflight during the administration of the composition.

In certain embodiments, the subject will (e.g., within an hour, within a day, within a week, within a month, within six months, within a year, within five years, or more) engage in spaceflight.

In certain embodiments, the subject has, may have, or may develop mitochondrial damage (e.g., mitochondrial dysfunction) (e.g., oxidative stress, inflammation) associated with spaceflight.

In certain embodiments, the subject has been subjected to or will be subjected to at least one or more of the following conditions (e.g., during spaceflight): stress, low gravity conditions, cosmic rays, or radiation.

In certain embodiments, the spaceflight-associated mitochondrial damage causes a loss of mitochondria in the subject (e.g., muscles of the subject).

In certain embodiments, the method further comprises isolating mitochondria.

In certain embodiments, the method comprises isolating mitochondria from a donor other than the subject.

In certain embodiments, the method comprises isolating mitochondria from the subject.

In certain embodiments, the method further comprises creating a cell bank (e.g., from a donor, from the subject).

In certain embodiments, the method comprises creating the cell bank from the subject prior to the subject engaging in spaceflight.

In certain embodiments, the cell bank comprises fibroblasts (e.g., primary fibroblasts) (e.g., primary fibroblasts from the subject) (e.g., primary fibroblasts from a donor).

In certain embodiments, the cell bank comprises mesenchymal stromal cells (MSCs) (e.g., MSCs from the subject) (e.g., MSCs from a donor).

In certain embodiments, the method comprises obtaining a tissue biopsy (e.g., a skin tissue biopsy) from the subject (e.g., prior to the subject experiencing spaceflight).

In certain embodiments, the method comprises creating a tissue bank (e.g., from a biopsy, e.g., a skin tissue biopsy)(e.g., a frozen tissue bank) from the subject.

In certain embodiments, the composition further comprises a mitochondrial storing buffer having a potassium ion concentration safe for administration to humans.

In certain embodiments, the administering step comprises parenterally administering at least one-unit dose of said composition to the subject.

In certain embodiments, the administering step comprises both intramuscular injection and intravenous injection of said composition to said subject.

In certain embodiments, isolating the mitochondria comprises preparing cell lysate from tissue via tissue dissociation.

In certain embodiments, isolating the mitochondria further comprises using a mitochondrial isolation buffer comprising a serine protease inhibitor.

In certain embodiments, the method further comprises isolating the mitochondria without using an antibiotic.

In certain embodiments, the subject and a donor from which mitochondria are obtained are not an HLA (human leukocyte antigen) match.

In certain embodiments, the composition administered to the subject does not comprise an antibiotic.

In certain embodiments, the composition comprises mitochondria isolated from human primary fibroblasts of a donor.

In certain embodiments, the composition comprises mitochondria isolated from mesenchymal stromal cells (MSCs) of a donor.

In certain embodiments, the isolating step is conducted using a mitochondrial isolation buffer composition.

In certain embodiments, the mitochondrial isolation buffer composition comprises: a buffering agent; a chelating agent; a sugar; an agent that acts as a membrane stabilizer and/or oxygen radical scavenger and/or binder of Ca²⁺ and/or binder of free fatty acid; and a serine protease inhibitor.

In certain embodiments, the mitochondrial isolation buffer composition does not comprise an antibiotic.

In certain embodiments, the method further comprises storing the isolated mitochondria at a temperature below −40° C.

In certain embodiments, the method further comprises administering to the subject an iron-chelating agent.

In certain embodiments, the method further comprises administering to the subject an antioxidant and/or a probiotic.

In certain embodiments, a mitochondrial isolation buffer composition comprises: a buffering agent; a chelating agent; a sugar; an agent that acts as a membrane stabilizer and/or oxygen radical scavenger and/or binder of Ca²⁺ and/or binder of free fatty acid; and a serine protease inhibitor.

In certain embodiments, the mitochondrial isolation buffer composition does not comprise an antibiotic.

In certain embodiments, a mitochondrial storing buffer composition comprises: one or more buffering agents; a source of magnesium ion; a chelating agent; a sugar; an antioxidant; a cytoprotective agent that binds to calcium ion; and an agent that acts as a membrane stabilizer and/or oxygen radical scavenger and/or binder of Ca²⁺ and/or binder of free fatty acid.

In certain embodiments, the mitochondrial storing buffer composition does not comprise an antibiotic.

In another aspect, the invention is directed to the use of autologous or allogeneic mitochondria for the treatment of spaceflight-associated mitochondrial damage (e.g., mitochondrial dysfunction) (e.g., in a method described herein) (e.g., using a composition described herein).

In another aspect, the invention is directed to the use of autologous or allogeneic mitochondria (e.g., in a method described herein) (e.g., using a composition described herein) for the treatment of spaceflight-associated mitochondrial damage (e.g., mitochondrial dysfunction), wherein the autologous or allogeneic mitochondria is administered to a subject suffering from spaceflight-associated mitochondrial damage.

In certain embodiments, the subject has experienced a spaceflight (e.g., a long-term spaceflight).

In certain embodiments, the spaceflight is at least a week (e.g., at least a month, at least 2 months, at least 3 months, at least 4 months, at least 6 months, at least a year, at least two years, or longer).

In certain embodiments, the subject is engaging in spaceflight (e.g., a long-term spaceflight).

In certain embodiments, the subject has been subjected to at least one or more of the following conditions (e.g., during spaceflight): stress, low gravity conditions, cosmic rays, or radiation.

In certain embodiments, the spaceflight-associated mitochondrial damage causes loss of mitochondria in the subject (e.g., muscles of the subject).

In certain embodiments, the spaceflight-associated mitochondrial damage comprises oxidative stress and/or inflammation.

In another aspect, the invention is directed to the use of autologous or allogeneic mitochondria for the treatment of spaceflight-associated mitochondrial damage, wherein the autologous or allogeneic mitochondria is administered to a subject prior to engaging in a spaceflight (e.g., within an hour, within a day, within a week, within a month, within six months, within a year, within five years, or more) (e.g., a long-term spaceflight).

In certain embodiments, the subject will experience a long-term spaceflight of at least a week (e.g., at least a month, at least 2 months, at least 3 months, at least 4 months, at least 6 months, at least a year, at least two years, or longer).

In certain embodiments, the subject will be subjected to at least one or more of the following conditions (e.g., during spaceflight): stress, low gravity conditions, cosmic rays, or radiation.

In certain embodiments, the spaceflight-associated mitochondrial damage causes loss of mitochondria in the subject (e.g., muscles of the subject).

In certain embodiments, the spaceflight-associated mitochondrial damage comprises oxidative stress and/or inflammation.

### Definitions

A or An: The articles “a” and “an” are used herein to refer to one or to more than one (i.e., to at least one) of the grammatical object of the article. By way of example, “an element” refers to one element or more than one element.
Administration: As used herein, the term “administration” typically refers to the administration of a composition to a subject or system, for example to achieve delivery of an agent that is, is included in, or is otherwise delivered by, the composition. Non-limiting examples of administration include oral administration; parenteral administration (for example, by subcutaneous, intramuscular, intravenous or epidural injection as, for example, a sterile solution or suspension, or sustained-release formulation, etc.); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.
Agent: As used herein, the term “agent” refers to an entity (e.g., for example, a cell, a component of a cell such as mitochondria or other organelle, a small molecule, a peptide, a polypeptide, a nucleic acid, a lipid, a polysaccharide, a complex, a combination, a mixture, a system, or a phenomenon such as heat, electric current, electric field, magnetic force, magnetic field, etc.).
Allogeneic: As used herein, the term “allogeneic” refers to any material derived from one subject (a donor) and transplanted to another subject. Examples of allogeneic transplantation include allogeneic T cell transplantation, allogeneic stem cell transplantation, and, as discussed herein, allogeneic mitochondrial transplantation.
Amelioration: As used herein, the term “amelioration” refers to the prevention, reduction, palliation, or improvement of a state of a subject. Amelioration includes, but does not require, complete recovery or complete prevention of a disease, disorder or condition.
Antibiotic: As used herein, the term “antibiotic” refers to an antibacterial substance such as penicillin, gentamicin, streptomycin, cephalosporin, ciprofloxacin, or the like, that is used to treat or prevent infections by killing or inhibiting the growth of bacterial in or on the body, that is administered orally, topically, or by injection, and that is isolated from cultures of certain microorganisms (such as fungi) or is of semi-synthetic or synthetic origin.
Biological Sample: As used herein, the term “biological sample” typically refers to a sample obtained or derived from a biological source (e.g., a tissue or organism or cell culture) of interest, as described herein. In some embodiments, e.g., as set forth herein, a biological source is or includes an organism, such as an animal or human. In some embodiments, e.g., as set forth herein, a biological sample is or include biological tissue or fluid. In some embodiments, e.g., as set forth herein, a biological sample can be or include cells, tissue (e.g., skin tissue, muscle, or other tissue), or bodily fluid. In some embodiments, e.g., as set forth herein, a biological sample can be or include blood, blood cells, cell-free DNA, free floating nucleic acids, ascites, biopsy samples, surgical specimens, cell-containing body fluids, sputum, saliva, feces, urine, cerebrospinal fluid, peritoneal fluid, pleural fluid, lymph, gynecological fluids, secretions, excretions, skin swabs, vaginal swabs, oral swabs, nasal swabs, washings or lavages such as a ductal lavages or bronchioalveolar lavages, aspirates, scrapings, bone marrow. In some embodiments, e.g., as set forth herein, a biological sample is or includes cells obtained from a single subject or from a plurality of subjects. A sample can be a “primary sample” obtained directly from a biological source, or can be a “processed sample.” A biological sample can also be referred to as a “sample.”
Improved, increased, or reduced: As used herein, these terms, or grammatically comparable comparative terms, indicate values that are relative to a comparable reference measurement. For example, in some embodiments, e.g., as set forth herein, an assessed value achieved with an agent of interest may be “improved” relative to that obtained with a comparable reference agent or with no agent. Alternatively or additionally, in some embodiments, e.g., as set forth herein, an assessed value in a subject or system of interest may be “improved” relative to that obtained in the same subject or system under different conditions or at a different point in time (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, e.g., as set forth herein, comparative terms refer to statistically relevant differences (e.g., differences of a prevalence and/or magnitude sufficient to achieve statistical relevance). Those of skill in the art will be aware, or will readily be able to determine, in a given context, a degree and/or prevalence of difference that is required or sufficient to achieve such statistical significance.
Isolated: As used herein, “isolated” refers to a substance and/or entity (e.g., including one or more mitochondria) that has been (a) separated from at least some of the components with which it was associated when initially produced (whether in nature, in a subject such as a donor, and/or in an experimental setting), and/or (b) designed, produced, prepared, and/or manufactured by the hand of man. Isolated substances and/or entities may be separated from at least about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated substances and/or entities are at least about 80%, about 85%, about 90%, about 91%, about 92%, about 93%, about 94%, about 95%, about 96%, about 97%, about 98%, about 99%, or more than about 99% pure. As used herein, a substance and/or entity is “pure” if it is substantially free of other components. In some embodiments, as will be understood by those skilled in the art, a substance and/or entity may still be considered “isolated” or “pure” after having been combined with certain other components such as, for example, one or more carriers or excipients (e.g., buffer, solvent, water, etc.); in such embodiments, percent isolation or purity of the substance and/or entity is calculated without including such carriers or excipients. To give but one example, in some embodiments, mitochondria that occur in nature can be referred to as “isolated” when, (a) they are present in a composition that does not include some or all of the components with which they were associated in nature, e.g., in a donor from which they were derived; (b) they are substantially free of other organelles of a donor organism from which they were derived; (c) they are present in a cell or system that differs from the donor organism from which they were derived. Thus, for instance, mitochondria removed from a donor for transplantation into a second different subject can be referred to as “isolated.”
Neurodegenerative disease: As used herein, the term “neurodegenerative disease” (also referred to as “degenerative nerve disease”) is an umbrella term for conditions which primarily affect the neurons in the human brain. In certain instances, neurodegenerative disease is characterized by a progressive loss of neurons associated with deposition of proteins showing altered physicochemical properties in the brain and/or in peripheral organs. Neurodegenerative diseases include, for example, amyotrophic lateral sclerosis (ALS), Parkinson's disease (PD) and PD-related disorders, Alzheimer's disease (AD), Lewy body dementia (LBD), other forms of dementia, muscular dystrophy (MD), mitochondrial disorders, prion diseases, motor neuron diseases (MND), Huntington's disease (HD), multiple sclerosis (MS), spinocerebellar ataxia (SCA), spinal muscular atrophy (SMA), Friedreich's ataxia, Batten disease, fatal familial insomnia, and others.
Pharmaceutical composition: As used herein, the term “pharmaceutical composition” refers to a composition in which an active agent is provided together with one or more pharmaceutically acceptable carriers. In some embodiments, e.g., as set forth herein, the active agent is present in a unit dose amount appropriate for administration to a subject, e.g., in a therapeutic regimen that shows a statistically significant probability of achieving a predetermined therapeutic effect when administered to a relevant population. In some embodiments, e.g., as set forth herein, a pharmaceutical composition can be formulated for administration in a particular form (e.g., in a solid form or a liquid form), and/or can be specifically adapted for, for example: oral administration (for example, as a drenche (aqueous or non-aqueous solutions or suspensions), tablet, capsule, bolus, powder, granule, paste, etc., which can be formulated specifically for example for buccal, sublingual, or systemic absorption); parenteral administration (for example, by subcutaneous, intramuscular, intravenous or epidural injection as, for example, a sterile solution or suspension, or sustained-release formulation, etc.); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.
Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.
Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation and/or modifies bioavailability of an agent, e.g., a pharmaceutical agent. Some examples of materials which can serve as pharmaceutically-acceptable carriers include: sugars, such as lactose, glucose and sucrose; starches, such as corn starch and potato starch; cellulose, and its derivatives, such as sodium carboxy methyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or poly anhydrides; and other non-toxic compatible substances employed in pharmaceutical formulations.
Prevent or prevention: The terms “prevent” and “prevention,” as used herein in connection with the occurrence of a disease, disorder, or condition, refers to reducing the risk of developing the disease, disorder, or condition; delaying onset of the disease, disorder, or condition; delaying onset of one or more characteristics or symptoms of the disease, disorder, or condition; and/or to reducing the frequency and/or severity of one or more characteristics or symptoms of the disease, disorder, or condition. Prevention can refer to prevention in a particular subject or to a statistical impact on a population of subjects. Prevention can be considered complete when onset of a disease, disorder, or condition has been delayed for a predefined period of time.
Prognosis: As used herein, the term “prognosis” refers to determining the qualitative or quantitative probability of at least one possible future outcome or event. As used herein, a prognosis can be a determination of the likely course of a disease, disorder, or condition such as cancer in a subject, a determination regarding the life expectancy of a subject, or a determination regarding response to therapy, e.g., to a particular therapy.
Reference: As used herein describes a standard or control relative to which a comparison is performed. For example, in some embodiments, e.g., as set forth herein, an agent, subject, animal, individual, population, sample, sequence, or value of interest is compared with a reference or control agent, subject, animal, individual, population, sample, sequence, or value. In some embodiments, e.g., as set forth herein, a reference or characteristic thereof is tested and/or determined substantially simultaneously with the testing or determination of the characteristic in a sample of interest. In some embodiments, e.g., as set forth herein, a reference is a historical reference, optionally embodied in a tangible medium. Typically, as would be understood by those of skill in the art, a reference is determined or characterized under comparable conditions or circumstances to those under assessment, e.g., with regard to a sample. Those skilled in the art will appreciate when sufficient similarities are present to justify reliance on and/or comparison to a particular possible reference or control.
Sample: As used herein, the term “sample” typically refers to an aliquot of material obtained or derived from a source of interest. In some embodiments, e.g., as set forth herein, a source of interest is a biological or environmental source. In some embodiments, e.g., as set forth herein, a sample is a “primary sample” obtained directly from a source of interest. In some embodiments, e.g., as set forth herein, as will be clear from context, the term “sample” refers to a preparation that is obtained by processing of a primary sample (e.g., by removing one or more components of and/or by adding one or more agents to a primary sample).
Susceptible to: An individual who is “susceptible to” a disease, disorder, or condition is at risk for developing the disease, disorder, or condition. In some embodiments, e.g., as set forth herein, an individual who is susceptible to a disease, disorder, or condition does not display any symptoms of the disease, disorder, or condition. In some embodiments, e.g., as set forth herein, an individual who is susceptible to a disease, disorder, or condition has not been diagnosed with the disease, disorder, and/or condition. In some embodiments, e.g., as set forth herein, an individual who is susceptible to a disease, disorder, or condition is an individual who has been exposed to conditions associated with, or presents a biomarker status associated with, development of the disease, disorder, or condition. In some embodiments, e.g., as set forth herein, a risk of developing a disease, disorder, and/or condition is a population-based risk (e.g., family members of individuals suffering from the disease, disorder, or condition).
Subject: As used herein, the term “subject” refers to an organism, typically a mammal (e.g., a human). In some embodiments, e.g., as set forth herein, a subject is suffering from a disease, disorder or condition. In some embodiments, e.g., as set forth herein, a subject is susceptible to a disease, disorder, or condition. In some embodiments, e.g., as set forth herein, a subject displays one or more symptoms or characteristics of a disease, disorder or condition. In some embodiments, e.g., as set forth herein, a subject is not suffering from a disease, disorder or condition. In some embodiments, e.g., as set forth herein, a subject does not display any symptom or characteristic of a disease, disorder, or condition. In some embodiments, e.g., as set forth herein, a subject is someone with one or more features characteristic of susceptibility to or risk of a disease, disorder, or condition. In some embodiments, e.g., as set forth herein, a subject is a patient. In some embodiments, e.g., as set forth herein, a subject is an individual to whom diagnosis has been performed and/or to whom therapy has been administered. In some instances, e.g., as set forth herein, a human subject can be interchangeably referred to as an “individual.”
Therapeutic agent, pharmaceutical agent, and active agent: As used herein, the terms “therapeutic agent”, “pharmaceutical agent”, and “active agent” are interchangeable, and each refers to any agent that elicits a desired pharmacological effect when administered to a subject. In some embodiments, e.g., as set forth herein, an agent is considered to be a therapeutic agent if it demonstrates a statistically significant effect across an appropriate population. In some embodiments, e.g., as set forth herein, the appropriate population can be a population of model organisms or a human population. In some embodiments, e.g., as set forth herein, an appropriate population can be defined by various criteria, such as a certain age group, gender, genetic background, preexisting clinical conditions, etc. In some embodiments, e.g., as set forth herein, a therapeutic agent is a substance that can be used for treatment of a disease, disorder, or condition. In some embodiments, e.g., as set forth herein, a therapeutic agent is an agent that has been or is required to be approved by a government agency before it can be marketed for administration to humans. In some embodiments, e.g., as set forth herein, a therapeutic agent is an agent for which a medical prescription is required for administration to humans.
Therapeutically effective amount: As used herein, the term “therapeutically effective amount” refers to an amount that produces a desired effect for which it is administered. In some embodiments, e.g., as set forth herein, the term refers to an amount that is sufficient, when administered to a population suffering from or susceptible to a disease, disorder, or condition, in accordance with a therapeutic dosing regimen, to treat the disease, disorder, or condition. Those of ordinary skill in the art will appreciate that the term therapeutically effective amount does not in fact require successful treatment be achieved in a particular individual. Rather, a therapeutically effective amount can be an amount that provides a particular desired pharmacological response in a significant number of subjects when administered to individuals in need of such treatment. In some embodiments, e.g., as set forth herein, reference to a therapeutically effective amount can be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent can be formulated and/or administered in a single dose. In some embodiments, e.g., as set forth herein, a therapeutically effective agent can be formulated and/or administered in a plurality of doses, for example, as part of a multi-dose dosing regimen.
Treatment: As used herein, the term “treatment” (also “treat” or “treating”) refers to administration of a therapy that partially or completely alleviates, ameliorates, relieves, inhibits, delays onset of, halts progression of, slows progression of, reverses progression of, reduces severity of, and/or reduces incidence of one or more symptoms, features, and/or causes of a particular disease, disorder, or condition, or is administered for the purpose of achieving any such result. In some embodiments, e.g., as set forth herein, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, e.g., as set forth herein, treatment can be of a subject who has been diagnosed as suffering from the relevant disease, disorder, and/or condition. In some embodiments, e.g., as set forth herein, treatment can be of a subject known to have one or more susceptibility factors that are statistically correlated with increased risk of development of the relevant disease, disorder, or condition. In various examples, treatment is of a cancer.
Unit dose: As used herein, the term “unit dose” refers to an amount administered as a single dose and/or in a physically discrete unit of a pharmaceutical composition. In many embodiments, e.g., as set forth herein, a unit dose contains a predetermined quantity of an active agent. In some embodiments, e.g., as set forth herein, a unit dose contains an entire single dose of the agent. In some embodiments, e.g., as set forth herein, more than one-unit dose is administered to achieve a total single dose. In some embodiments, e.g., as set forth herein, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic agent(s). For example, acceptable carriers (e.g., pharmaceutically acceptable carriers), diluents, stabilizers, buffers, preservatives, etc., can be included. It will be appreciated by those skilled in the art, in many embodiments, e.g., as set forth herein, a total appropriate daily dosage of a particular therapeutic agent can comprise a portion, or a plurality, of unit doses, and can be decided, for example, by a medical practitioner within the scope of sound medical judgment. In some embodiments, e.g., as set forth herein, the specific effective dose level for any particular subject or organism can depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts
Variant: As used herein, the term “variant” refers to an entity that shows significant structural identity with a reference entity but differs structurally from the reference entity in the presence, absence, or level of one or more chemical moieties as compared with the reference entity. In some embodiments, e.g., as set forth herein, a variant also differs functionally from its reference entity. In general, whether a particular entity is properly considered to be a “variant” of a reference entity is based on its degree of structural identity with the reference entity. A variant can be a molecule comparable, but not identical to, a reference. For example, a variant peptide can differ from a reference peptide at one or more differences in the amino acid sequence. In some embodiments, e.g., as set forth herein, a variant peptide shows an overall sequence identity with a reference peptide that is at least 85%, 86%, 87%, 88%, 89%, 90%, 91%, 92%, 93%, 94%, 95%, 96%, 97%, or 99%.

### DESCRIPTION OF THE INVENTION

Incorporated herein is U.S. Provisional Patent Application No. 63/405,336, filed Sep. 9, 2022, entitled, “Compositions and Methods for Treatment of Traumatic Brain Injury (TBI), for Example, Mild Traumatic Brain Injury (mTBI)”, which describes compositions and methods involving mitochondrial organelle transplantation for use in the treatment of traumatic brain injury (TBI), e.g., mild traumatic brain injury (mTBI), in a subject. Also incorporated herein is International (PCT) Patent Application No. PCT/US2020/047359, filed Aug. 21, 2020, and published as International Publication No. WO 2021/141637, which describes compositions and methods for treatment of amyotrophic lateral sclerosis (ALS); other neurodegenerative diseases (ND) such as Alzheimer's disease, Parkinson's disease, muscular dystrophy; and other mitochondrial disorders. In particular, described therein are experimental examples demonstrating mitochondrial organelle transplantation™ (MOT™) for the treatment of NDs such as ALS. This application incorporates by reference U.S. patent application Ser. No. 16/937,388, filed on Jul. 23, 2020, and U.S. Provisional Patent Application No. 62/958,592, filed Jan. 8, 2020. The contents of each of the above-referenced patent applications are incorporated by reference herein in their entirety. Moreover, all publications mentioned herein are incorporated by reference herein in their entirety.

In recent years, mitochondrial transplantation (MT) has shed a new light of therapeutic intervention that benefits neuronal survival and regeneration for neurodegenerative diseases, stroke and central nerve system (CNS) injury. Human mitochondria from primary fibroblasts have been transplanted into mouse motor neuronal NSC-34 mitochondrial DNA (mtDNA)-depleted (p0) cells. MT replenishes mtDNA and restores mitochondrially encoded cytochrome c oxidase 1 (MT-CO1 ) and mitochondrially encoded NADH dehydrogenase 1 (MT-ND1) and rescues aerobic respiration of NSC-34 ρ0 cells.

Shi et al., intravenously injected labeled xenogeneic mitochondria from human hepatoma into the experimental Parkinson's disease (PD) model rat. In Shi, the isolated mitochondria remained stable in serum for at least 2 hours. After intravenous injection into the model rat, the labeled mitochondria was widely distributed into various tissues including brain tissue. However, red blood cells did not uptake mitochondria after intravenous injection. The mitochondrial transplantation described in Shi et al. prevented experimental PD progress through increasing activity of electron transport chain, decreasing ROS level, and preventing cell apoptosis and necrosis.

Another group, Chang et al., reported the internalization of exogenous mitochondria to recipient cells lasted 15 days and rescued mitochondrial function and prevented mitochondria dependent cell death. Mitochondrial biogenesis genes were also upregulated by the exogenous mitochondria for several days. Exogenous mitochondria began to enter recipient cells within minutes. The exogenous mitochondria successfully progressed through the endolysosomal system. Some of these mitochondria escape from endosomal and lysosomal compartments and effectively fuse with endogenous mitochondria. Several proteins, including MFN1 and MFN2 in mitochondrial outer membrane and OPA1 in inner membrane, are involved in fusion of exogenous mitochondria with endogenous mitochondria in recipient cells. Cells in vitro and in vivo are capable of uptaking mitochondria by different and active mechanisms such as endocytosis, macropinocytosis and actin-dependent internalization. Allogeneic mitochondria induced Treg and promoted immune regulation, were not immunogenic.

### A. Mitochondria

In eukaryotic cells, mitochondria are known as the powerhouse, which generates adenosine triphosphate (ATP), by oxidative phosphorylation (OXPHOS). They also play an important role in synthesis of iron-sulfur clusters and heme, β-oxidation of fatty acids, homeostasis of calcium, iron and reactive oxygen species (ROS). Mitochondria are of particular importance in neurons. Neurons have high metabolic requirements—the brain consumes 20% of the body's resting ATP production despite being only 2% of its mass. Moreover, mitochondria are essential calcium buffering organelles in neurons that modulate local calcium dynamics, for example, regulate neurotransmitter release. Neurons are long-lived cells that persist throughout the lifespan of the individual and as such are more susceptible to the accumulating damage arising from mitochondrial dysfunction. Severe mitochondrial dysfunction comes in many forms, including defective OXPHOS, excessive ROS, impaired calcium buffering capacity, and defective mitochondrial dynamics

In eukaryotic cells, mitochondria generate ATP by oxidative phosphorylation (OXPHOS) in the presence of oxygen. Mitochondria also play an important role in synthesis of iron-sulfur (Fe—S) clusters, β-oxidation of fatty acids, synthesis of heme prosthetic groups, the urea cycle, as well as homeostasis of calcium, iron and reactive oxygen species (ROS). Mitochondria are highly dynamic organelles which frequently fuse and divide. Mitochondrial fusion/fission allow segregation of damaged mitochondria, mitophagy to remove damaged mitochondria, and ultimately cell death if the damage is too severe. In addition, mitochondria can transfer between cells. Cells may be able to obtain functional mitochondria from other cells in order to satisfy their bioenergetics and biosynthetic needs. Without wishing to be bound to any particular theory, the possible mechanisms include tunneling nanotubes, extracellular vesicles and partial or complete cell fusion.

Mitochondrial dysfunction contributes to many diseases such as neurodegenerative disease, cardiac disease, and cancer. Mitochondrial dysfunction broadly includes states in which mitochondria of a cell, tissue, organism, or sample thereof, are characterized by (1) a decreased rate, amount, or efficiency of ATP production; (2) a decreased mitochondrial membrane potential; (3) a decreased number or concentration of mitochondria; and/or (4) an increased rate or amount of ROS production, relative to a reference. In some embodiments, a reference is a measurement or value representative of a healthy subject (e.g., a comparable subject with typical mitochondrial function and/or without a diagnosed medical condition known to impact mitochondrial function). In some embodiments, a reference is a measurement or value representative of a population of healthy subjects. In some embodiments, a reference is a measurement or value representative of the subject at an earlier time. Methods and techniques for measuring mitochondrial ATP production, mitochondrial membrane potential, number or concentration of mitochondria, and/or ROS production are known in the art.

Mitochondrial dysfunction has been documented in amyotrophic lateral sclerosis (ALS), Alzheimer's disease (AD), and Parkinson's disease (PD), for example. Mitochondria are essential for neural function because neurons highly depend on aerobic OXPHOS in mitochondria for their energetic needs. Defective mitochondrial respiration and ATP production in neurons result in neural dysfunction and degeneration. Mitochondria also produce ROS. If oxidative stress of ROS overwhelms the antioxidative defense most from superoxide dismutase (SOD), catalase (CAT) and glutathione peroxidase (GPX), ROS causes protein, lipid and DNA damage of neurons. In addition, overload of mitochondrial calcium and iron impairs ATP production and structures of mitochondria and neurons.

Mitochondria are highly present in cardiac cells due to the increased energy demands of such cells. Mitochondrial dysfunction is associated with the development of numerous cardiac diseases such as atherosclerosis, ischemia-reperfusion injury, hypertension, cardiac hypertrophy and heart failure.

Defects in mitochondrial function have also been linked to tumorigenesis. For example, it has been observed that cancer cells have an increase in glycolysis and lactate production in the presence of oxygen without an increase in OXPHOS, known as the “Warburg Effect”. Many cancers have mitochondrial defects and dysfunction. Glycolytic inhibitors have been found to suppress tumor growth in animal models and clinical trials.

### B. Long Term Spaceflight

Mitochondria of a subject can be affected (e.g., damaged) due to a spaceflight (e.g., travel through outer space) and travel to other interstellar bodies (e.g., planets, asteroids, moons). In certain embodiments, space is at least 50 miles above Earth's sea level. In certain embodiments, space is at least 62 miles (100 km) above Earth's sea level. In certain embodiments, space is at least 100 miles, at least 125 miles, at least 150 miles, at least 200 miles, at least 250 miles, at least 300 miles or more above Earth's sea level.

In certain embodiments, a subject treated using a composition described herein experiences a long-term space flight. In certain embodiments, a long-term spaceflight is a period of time during which a subject remains substantially in space either continuously or with minimal interruption. In certain embodiments, a long-term spaceflight is at least a week (e.g., at least a month, at least 2 months, at least 3 months, at least 4 months, at least 6 months, at least a year, at least two years, or longer).

In certain embodiments, a subject will (e.g., within an hour, within a day, within a week, within a month, within six months, within a year, within five years, or more) engage in spaceflight after receiving a composition described herein. In certain embodiments, subject is engaging in spaceflight during the administration of a composition described herein.

In certain embodiments, a subject who remains in space for a period of time may experience adverse health conditions resulting in or resulting from mitochondrial damage due to being subjected to environmental conditions of outer space and/or spaceflight (e.g., long-term spaceflight). In certain embodiments, environmental conditions of outer space include, but are not limited to, low gravity conditions (e.g., micro-gravity, zero gravity), cosmic rays (e.g., cosmic radiation) and radiation. In certain embodiments, spaceflight may result in or contribute to sleep deprivation and muscle weakness (e.g., due to low gravity conditions). In certain embodiments, spaceflight-associated mitochondrial damage comprises oxidative stress and/or inflammation. In certain embodiments, adverse health conditions include mitochondrial loss or depletion (e.g., from tissue of a subject).

### C. Selection of Tissue Donors

Where allogeneic mitochondria are used (e.g., from a donor other than the subject receiving the mitochondria), prior to donation of a tissue (e.g., skin) to a cell bank (e.g., a fibroblast cell bank) for isolation of mitochondria, a donor is screened. In certain embodiments, screening a donor ensures that they are suitable for donation of tissue (e.g., skin tissue) from which fibroblasts are obtained. Mitochondria are isolated from cells obtained from a donor.

In certain embodiments, a donor is selected as required by FDA 21 CFR Part 1271, which is incorporated by reference in its entirety. In certain embodiments, a donor is screened for one or more diseases (e.g., communicable diseases, e.g., a virus). In certain embodiments, a donor is free from risk factors for, and clinical evidence of, infection due to a relevant communicable disease agent and/or disease. In certain embodiments, a donor is free from communicable disease risks associated with xenotransplantation. In certain embodiments, a donor is tested for communicable disease agents and is determined to be negative or nonreactive. In certain embodiments, a donor is free from diseases including, but not limited to, human immunodeficiency virus (HIV), hepatitis B virus, hepatitis C virus, human transmissible spongiform encephalopathy (e.g., Creutzfeldt-Jakob disease), treponema pallidum, and communicable diseases of the genitourinary tract (e.g., chlamydia trachomatis, neisseria gonorrhea).

In certain embodiments, a donor does not have a neurodegenerative disease and/or other condition associated with mitochondrial dysfunction. In certain embodiments, a donor does not have a disease including, but not limited to, amyotrophic lateral sclerosis (ALS), Parkinson's disease (PD), a PD related disorder, Alzheimer's disease (AD), Lewy body dementia (LBD), dementia, muscular dystrophy (MD), a mitochondrial disorder, prion disease, motor neurone disease (MND), Huntington's disease (HD), multiple sclerosis (MS), spinocerebellar ataxia (SCA), spinal muscular atrophy (SMA), Friedreich's ataxia, Batten disease, and fatal familial insomnia.

Mitochondria derived from primary fibroblasts exhibit low or no immunogenicity and, accordingly, need no HLA tying match in mitochondrial transplantation. Humans have three main MHC class I loci, known as HLA-A, HLA-B, and HLAC, each individual carrying two alleles at each locus. Humans have six main MHC class II loci, known as HLA-DPA1, HLA-DPB1, HLA-DQA1, HLA-DQB1, HLA-DRA, and HLA-DRB1, each individual carrying two alleles at each locus. In general, a donor and a subject would be matched based on alleles present in the donor and the subject at one or more HLA loci, such as HLA-A, HLA-B, HLA-C, HLA-DRB1, HLA-DQB1, and/or HLA-DPB1. Various standards for HLA matching are known in the art. Matching of all 8 alleles at HLA-A, HLA-B, HLA-C, and HLA-DRB 1 loci can be referred to as an 8/8 match. Matching of all 10 alleles at HLA-A, HLAB, HLA-C, HLA-DRB 1, and HLA-DQB 1 loci can be referred to as a 10/10 match. For certain transplantations, varying degrees of allele mismatch can be accepted. Thus, for example, a donor and a subject can be matched, e.g., at 10, 9, 8, 7, 6, 5, 4, 3, 2, 1, or 0 alleles of 10 at HLA-A, HLA-B, HLA-C, HLA-DRB1, and HLA-DQB1, or at 8, 7, 6, 5, 4, 3, 2, 1, or 0 alleles of 8 HLAA, HLA-B, HLA-C, and HLA-DRB1.

As is disclosed herein, in various embodiments, a mitochondrial donor and a subject of mitochondrial transplantation or treatment do not need to be an HLA (human leukocyte antigen) match [e.g., not an identical match (e.g., based on match of 8 or 10 tested HLA markers) and/or not a haploidentical match (e.g., based on match of 8 or 10 tested HLA markers), and/or of indeterminate match status (e.g., no HLA markers tested prior to the administering step). This is a significant advantage over stem cell therapy and organ transplantation which require HLA matching of donor and recipient.

### D. Obtaining Cells From Donor Tissue

In some embodiments, a cell bank is created from cells of a donor (e.g., a donor different from the subject receiving a mitochondrial composition) or a subject (e.g., a subject prior to engaging in space flight), which is used as a source to obtain isolated mitochondria. In some embodiments, a cell bank is a frozen cell bank. In some embodiments, cells are stored in a cell bank by freezing. In certain embodiments, cells are frozen at a temperature below −40° C. (e.g., below −60° C., below −70° C., below −80° C., e.g., using liquid nitrogen). In certain embodiments, medium used to freeze cells to create a cell bank does not contain antibiotics (e.g., gentamicin, penicillin, streptomycin).

In some embodiments, skin tissue is used as source material to create a cell bank. In certain embodiments, fibroblast cells from skin tissue are obtained (e.g., surgically, e.g., using a tissue biopsy) to create a cell bank. In certain embodiments, fibroblast cells from skin tissue are expanded prior to freezing. In certain embodiments, fibroblast cells from skin tissue are expanded in a culture media that is not supplemented with antibiotics (e.g., gentamicin, penicillin, streptomycin). Antibiotics in culture media can be toxic to mitochondria of mammalian cells. In certain embodiments, fibroblast cells from a first, a second, or a third passage are frozen to create a cell bank.

A person of skill in the art would be able to select appropriate freezing protocols corresponding to tissue and cell types described herein based on information provided in the present disclosure.

### E. Mitochondrial Isolation and Storing Buffers

In certain embodiments, the methods and compositions described herein utilize isolation and storing buffers for mitochondria. In certain embodiments, mitochondrial isolation and storing buffers used are described in International (PCT) Patent Application No. PCT/US2020/047359, filed Aug. 21, 2020, which is incorporated by reference in its entirety.

In certain embodiments, an isolation buffer comprises a buffering agent [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an agent that acts as a membrane stabilizer and/or oxygen radical scavenger and/or binder of Ca²⁺ and/or binder of free fatty acid (e.g., bovine serum albumin, BSA); and a serine protease inhibitor (e.g., phenylmethylsulfonyl fluoride (PMFS), also called phenylmethane sulfonyl fluoride). In certain embodiments, the composition further comprises isolated donor mitochondria, e.g., fibroblast mitochondria.

In certain embodiments, a mitochondrial isolation buffer is comprised of 300 mM sucrose, 10 mM K-HEPES, 1 mM K-EGTA, 0.1%BSA and 0.25 mM PMSF (Sigma Aldrich, St Louis, MO, USA). In certain embodiments, the osmolarity of the buffer is about 325 mOsm. In certain embodiments, the concentration of potassium ion is 11 mM. Bovine serum albumin (BSA) is a membrane stabilizer, oxygen radical scavenger, and binds Ca²⁺ and free fatty acids. Phenylmethylsulfonyl fluoride (PMSF), also called phenylmethane sulfonyl fluoride, is a serine protease inhibitor used in the preparation of cell lysates. Lysosomes are organelles that contain digestive enzymes which digest excess or worn out organelles. During the procedure of cell homogenization (e.g., to obtain mitochondria), some lysosomes may be damaged and release the digestive enzymes to the cell lysate. In certain embodiments, in order to prevent the damage of mitochondria from the digestive enzymes, PMSF can be included in the isolation buffer. In certain embodiments, the isolation buffer does not contain antibiotics.

In certain embodiments, a mitochondrial storage buffer is administered to a subject and/or used to maintain isolated mitochondria stably in solution. In certain embodiments, a storage buffer comprises one or more buffering agents [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)] [e.g., monopotassium phosphate (KH₂PO₄)]; a source of magnesium ion [e.g., magnesium chloride (MgCl₂)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an antioxidant [e.g., taurine]; a cytoprotective agent that binds to calcium ion [e.g., lactobionate or salt thereof, e.g., K-lactobionate]; and an agent that acts as a membrane stabilizer and/or oxygen radical scavenger and/or binder of Ca²⁺ and/or binder of free fatty acid (e.g., bovine serum albumin, BSA). In certain embodiments, the composition further comprises isolated donor mitochondria (e.g., fibroblast mitochondria).

In certain embodiments, a mitochondrial storing buffer is comprised of 240 mM sucrose, 2 mM KH₂PO₄, 3 mM MgCl₂, 10 mM K-HEPES, 20 mM Taurine, 1 mM K-EGTA, 0.1% BSA and 15 mM K-lactobionate (Sigma Aldrich, St Louis, MO, USA). Taurine acts as an antioxidant that scavenges free radical species generated by mitochondria, and is also involved to membrane stabilization, osmoregulation and ion channel regulation. Lactobionate has cytoprotective property and prevents mitochondrial swelling. Lactobionate also binds to calcium ion with high affinity and acts as a calcium chelator. In certain embodiments, the osmolarity of a mitochondrial storing buffer is about 325 mOsm. In certain embodiments, a mitochondrial storing buffer contains about 28 mM potassium ion. In certain embodiments, buffers are sterilized by filtering (e.g., through 0.22 μm filter), aliquoted to small vials and stored at −80° C.

In certain embodiments (e.g., clinical uses discussed herein), mitochondria in the storing buffer is administered (e.g., intramuscularly, intravenously, subcutaneously) (e.g., both intramuscularly and intravenously) to a human subject. High potassium ion concentrations are dangerous for injection to humans (e.g., 91 mM potassium ion concentration). In certain embodiments, a reduced concentration of all K⁺ salts is used in a mitochondrial storing buffer (e.g., 2 mM KH₂PO₄, 10 mM K-HEPES, and 15 mM K-lactobionate). In certain embodiments, the final concentration of K⁺ in a storing buffer solution used is similar to clinical intravenous solution with potassium chloride (e.g., about 28 mEq) (e.g., from 20 mEq to 40 mEq). To keep the osmolarity of the storing buffer at a desired level, the concentration of sucrose can be increased in the storing buffer. In certain embodiments, the storing buffer is not administered along with antibiotics.

### F. Constructive Example

In the present, non-limiting example, Applicant proposes that transplantation of autologous or allogeneic mitochondria will restore mitochondrial function and alleviate oxidative stress and inflammation of astronauts after long-term spaceflight.

In certain embodiments, a cell bank (e.g., a fibroblast cell bank) is developed for use in production and isolation of mitochondria for transplantation (MT). The cell bank uses cells obtained either from a donor other than the subject to be treated (e.g., astronaut) or from the subject himself.

In an embodiments, where autologous mitochondria are used, astronauts may have a skin biopsy before spaceflight. A fibroblast bank is developed from their own skin tissues.

In certain embodiments, where allogeneic mitochondria are used, skin donors are selected as requirement by FDA 21 CFR Part 1271, which is incorporated by reference in its entirety. A fibroblast bank is developed from cells obtained from selected skin donors.

Fibroblasts from the cell bank are expanded. Mitochondria are isolated from the fibroblasts. In certain embodiments, fibroblasts are cultured and mitochondria isolated as described herein. In certain embodiments, fibroblasts are cultured and mitochondria are isolated as described in PCT Application No. PCT/US2020/047359, filed Aug. 21, 2020.

After a long-term spaceflight, an astronaut is injected intravenously with a dose of mitochondria, for example, from 50-100 million fibroblasts. Patients are monitored clinically and via laboratory studies.

In certain embodiments, fibroblasts are the type of cells used for isolating mitochondria for transplantation. In other embodiments, other cell types may be used to generate viable mitochondria for transplantation, for example, mesenchymal stromal cells (MSCs).

The source of mitochondrial damage experienced by astronauts is unknown but may include, for example, stress, low gravity conditions, cosmic rays, radiation, sleep deprivation, and/or other factors.

### Publications

1. Nikawa, T., Ishidoh, K., Hirasaka, K., Ishihara, I., Ikemoto, M., Kano, M., Kominami, E., Nonaka, I., Ogawa, T., Adams, G.R., et al. (2004). Skeletal muscle gene expression in space-flown rats. FASEB J. 18, 522-524.
2. Klimchuk, D.O. (2007). Structural and functional features of mitochondria in statocytes of soybean root under microgravity conditions. Cytol. Genet. 41, 25-29.
3. Smith, R.W., Scott, P.J., and Szpunar, B. (2009). Solute diffusion in nonionic liquids-effects of gravity. Ann. N Y Acad. Sci. 1161, 526-536.
4. Silveira, W.A., Fazelinia, K., Rosenthal, S.B., Mason, C.E., Costes, S.V., Beheshti, A., et al (2020). Comprehensive Multi-omics Analysis Reveals Mitochondrial Stress as a Central Biological Hub for Spaceflight Impact. Cell 183(5),1185-1201. DOI:https://doi.org/10.1016/j.cell.2020.11.002.
5. Indo, H.P., Majima, H.J., Terada, T., Suenaga, S., Tomita, K., Yamada, S., et al. (2016). Changes in mitochondrial homeostasis and redox status in astronauts following long stays in space. Sci Rep 6:39015. doi: 10.1038/srep39015.
6. Jiang XP, Baucom CC, Elliott RL. (2020). Mitochondria dynamically transplant into cells in vitro and in mice and rescue aerobic respiration of mitochondrial DNA-depleted motor neuron NSC-34. J. Biomedical Sci. Engineering. 13:203-221. doi: 10.4236/jbise.2020.139019.
7. Shi X, Zhao M, Fu C, Fu A. (2017) Intravenous administration of mitochondria for treating experimental Parkinson's disease. Mitochondrion 34:91-100. https://doi.org/10.1016/j.mito.2017.02.005
8. Chang J.C., Liu K.H., Li Y.C., Kou S.J., Wei Y.H., Chuang C. S, et al. (2013). Functional Recovery of Human Cells Harbouring the Mitochondrial DNA Mutation MERRF A8344G via Peptide-Mediated Mitochondrial Delivery. Neurosignals 21:160-173.
9. Douglas B Cowan, Rouan Yao, Jerusha K Thedsanamoorthy, David Zurakowski, Pedro J Del Nido, James D McCully (2017) Transit and integration of extracellular mitochondria in human heart cells. Sci. Rep. 7(1):17450. doi: 10.1038/s41598-017-17813-0.
10. Tomoya Kitani, Daisuke Kami, Satoaki Matoba, Satoshi Gojo (2014). Internalization of isolated functional mitochondria: involvement of macropinocytosis. J. Cell. Mol. Med. 18(8)1694-1703. https://doi.org/10.1111/jcmm.12316
11. Luz-Crawford, P., et al. (2019). Mesenchymal stem cell repression of Th17 cells is triggered by mitochondrial transfer. Stem Cell Res Ther. 10(1):232. doi: 10.1186/s13287-019-1307-9.
12. Ramirez-Barbieri, G., et al. (2019) Alloreactivity and allorecognition of syngeneic and allogeneic mitochondria. Mitochondrion 46:103-115. doi: 10.1016/j.mito.2018.03.002

It is contemplated that systems, compositions, methods, and processes of the claimed invention encompass variations and adaptations developed using information from the embodiments described herein. Adaptation and/or modification of the systems, compositions, methods, and processes described herein may be performed, as contemplated by this description.

Throughout the description, where articles, compositions, and systems are described as having, including, or comprising specific components, or where processes and methods are described as having, including, or comprising specific steps, it is contemplated that, additionally, there are articles, compositions, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein.

Documents are incorporated herein by reference as noted. Where there is any discrepancy in the meaning of a particular term, the meaning provided in this document is controlling.

Headers are provided for the convenience of the reader-the presence and/or placement of a header is not intended to limit the scope of the subject matter described herein.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A method for autologous or allogeneic transplantation of mitochondria in a subject for treatment and/or prevention and/or amelioration of spaceflight-associated mitochondrial damage, said method comprising administering to said subject a composition comprising mitochondria isolated from the subject or isolated from a donor other than the subject, wherein (i) the subject has engaged in spaceflight or is engaging in spaceflight at the time of administration of the composition and has or may have mitochondrial damage associated with said spaceflight, or (ii) wherein the subject is about to engage in spaceflight at the time of administration of the composition and said composition is administered to prevent or ameliorate spaceflight-associated mitochondrial damage.

2. The method of claim 1, wherein the composition further comprises a mitochondrial storing buffer having a potassium ion concentration safe for administration to humans.

3. The method of claim 1, wherein the administering step comprises parenterally administering at least one-unit dose of said composition to said subject.

4. The method of claim 3, wherein the administering step comprises both intramuscular injection and intravenous injection of said composition to said subject.

5. The method of claim 1, further comprising (i) isolating said mitochondria from said donor other than the subject or (ii) isolating said mitochondria from said subject.

6. The method of claim 5, wherein isolating said donor mitochondria comprises preparing cell lysate from tissue of the donor via tissue dissociation.

7. The method of claim 5, wherein isolating said donor mitochondria comprises using a mitochondrial isolation buffer comprising a serine protease inhibitor.

8. The method of claim 5, comprising isolating said donor mitochondria without using an antibiotic.

9. The method of claim 1, wherein the donor and the subject are not an HLA (human leukocyte antigen) match.

10. The method of claim 1, wherein the composition administered to the subject does not comprise an antibiotic.

11. The method of claim 1, wherein the composition comprises mitochondria isolated from human primary fibroblasts of the donor.

12. The method of claim 1, wherein the composition comprises mitochondria isolated from mesenchymal stromal cells (MSCs) of the donor (or of the pre-spaceflight subject).

13. The method of claim 1, further comprising isolating the mitochondria from the donor (or of the pre-spaceflight subject), wherein the isolating step is conducted using a mitochondrial isolation buffer composition.

14. The method of claim 13, wherein the mitochondrial isolation buffer composition comprises:
a buffering agent;
a chelating agent;
a sugar;
an agent that acts as a membrane stabilizer and/or oxygen radical scavenger and/or binder of Ca²⁺ and/or binder of free fatty acid; and
a serine protease inhibitor.

15. The mitochondrial isolation buffer composition of claim 14, wherein the composition does not comprise an antibiotic.

16. The method of claim 12, further comprising storing the isolated mitochondria at a temperature below −40° C.

17. The method of claim 1, comprising administering to the subject an iron-chelating agent.

18. The method of claim 1, comprising administering to the subject an antioxidant and/or a probiotic.

19. A mitochondrial isolation buffer composition for use in performing the method of claim 1, said composition comprising:
a buffering agent;
a chelating agent;
a sugar;
an agent that acts as a membrane stabilizer and/or oxygen radical scavenger and/or binder of Ca²⁺ and/or binder of free fatty acid; and
a serine protease inhibitor.

20. The mitochondrial isolation buffer composition of claim 19, wherein the composition does not comprise an antibiotic.

21. A mitochondrial storing buffer composition for use in performing the method of claim 1, said composition comprising:
one or more buffering agents;
a source of magnesium ion;
a chelating agent;
a sugar;
an antioxidant;
a cytoprotective agent that binds to calcium ion; and
an agent that acts as a membrane stabilizer and/or oxygen radical scavenger and/or binder of Ca²¹ and/or binder of free fatty acid.

22. The mitochondrial storing buffer composition of claim 21, wherein the composition does not comprise an antibiotic.

23. A kit comprising a donor mitochondria composition in a unit dosage effective to treat and/or ameliorate, and/or prevent spaceflight-associated mitochondrial damage in a subject, said donor mitochondria composition comprising:
mitochondria isolated from tissue of a donor;
one or more buffering agents;
a source of magnesium ion;
a chelating agent;
a sugar;
an antioxidant;
a cytoprotective agent that binds to calcium ion; and
an agent that acts as a membrane stabilizer and/or oxygen radical scavenger and/or binder of Ca²⁺ and/or binder of free fatty acid.

24. The kit of claim 23, wherein the donor and the subject are not an HLA (human leukocyte antigen) match.

25. The kit of claim 23, wherein the donor mitochondria composition does not comprise an antibiotic.

26. The kit of claim 23, further comprising instructions for optimizing the dose and/or frequency and/or route of administration of the composition.

27. A method of treating spaceflight-associated mitochondrial damage, the method comprising:
administering to a subject a composition comprising mitochondria, wherein the mitochondria is autologous or allogeneic mitochondria.

28. The method of claim 27, wherein the subject has engaged in spaceflight prior to the administration of the composition.

29. The method of claim 27, wherein the subject is engaging in spaceflight during the administration of the composition.

30. The method of claim 27, wherein the subject will engage in spaceflight.

31. The method of claim 27, wherein the subject has, may have, or may develop mitochondrial damage associated with spaceflight.

32. The method of claim 31, wherein the subject has been subjected to or will be subjected to at least one or more of the following conditions: stress, low gravity conditions, cosmic rays, or radiation.

33. The method of claim 31, wherein the spaceflight-associated mitochondrial damage causes a loss of mitochondria in the subject.

34. The method of claim 27, wherein the method further comprises isolating mitochondria.

35. The method of claim 34, wherein the method comprises isolating mitochondria from a donor other than the subject.

36. The method of claim 34, wherein the method comprises isolating mitochondria from the subject.

37. The method of claim 27, wherein the method further comprises creating a cell bank.

38. The method of claim 37, wherein the method comprises creating the cell bank from the subject prior to the subject engaging in spaceflight.

39. The method of claim 37, wherein the cell bank comprises fibroblasts.

40. The method of claim 37, wherein the cell bank comprises mesenchymal stromal cells (MSCs).

41. The method of claim 27, wherein the method comprises obtaining a tissue biopsy from the subject.

42. The method of claim 27, wherein the method comprises creating a tissue bank from the subject.

43. The method of claim 27, wherein the composition further comprises a mitochondrial storing buffer having a potassium ion concentration safe for administration to humans.

44. The method of claim 27, wherein the administering step comprises parenterally administering at least one-unit dose of said composition to the subject.

45. The method of claim 44, wherein the administering step comprises both intramuscular injection and intravenous injection of said composition to said subject.

46. The method of claim 37, wherein isolating the mitochondria comprises preparing cell lysate from tissue via tissue dissociation.

47. The method of claim 37, wherein isolating the mitochondria comprises using a mitochondrial isolation buffer comprising a serine protease inhibitor.

48. The method of claim 37, comprising isolating the mitochondria without using an antibiotic.

49. The method of claim 27, wherein the subject and a donor from which mitochondria are obtained are not an HLA (human leukocyte antigen) match.

50. The method of claim 27, wherein the composition administered to the subject does not comprise an antibiotic.

51. The method of claim 27, wherein the composition comprises mitochondria isolated from human primary fibroblasts of a donor.

52. The method of claim 27, wherein the composition comprises mitochondria isolated from mesenchymal stromal cells (MSCs) of a donor.

53. The method of claim 37, wherein the isolating step is conducted using a mitochondrial isolation buffer composition.

54. The method of claim 53, wherein the mitochondrial isolation buffer composition comprises:
a buffering agent;
a chelating agent;
a sugar;
an agent that acts as a membrane stabilizer and/or oxygen radical scavenger and/or binder of Ca²⁺ and/or binder of free fatty acid; and
a serine protease inhibitor.

55. The mitochondrial isolation buffer composition of claim 54, wherein the composition does not comprise an antibiotic.

56. The method of claim 37, further comprising storing the isolated mitochondria at a temperature below −40° C.

57. The method of claim 27, comprising administering to the subject an iron-chelating agent.

58. The method of claim 27, comprising administering to the subject an antioxidant and/or a probiotic.

59. A mitochondrial isolation buffer composition for use in performing the method of claim 27, said composition comprising:
a buffering agent;
a chelating agent;
a sugar;
an agent that acts as a membrane stabilizer and/or oxygen radical scavenger and/or binder of Ca²⁺ and/or binder of free fatty acid; and
a serine protease inhibitor.

60. The mitochondrial isolation buffer composition of claim 59, wherein the composition does not comprise an antibiotic.

61. A mitochondrial storing buffer composition for use in performing the method of claim 27, said composition comprising:
one or more buffering agents;
a source of magnesium ion;
a chelating agent;
a sugar;
an antioxidant;
a cytoprotective agent that binds to calcium ion; and
an agent that acts as a membrane stabilizer and/or oxygen radical scavenger and/or binder of Ca²⁺ and/or binder of free fatty acid.

62. The mitochondrial storing buffer composition of claim 61, wherein the composition does not comprise an antibiotic.

63. Use of autologous or allogeneic mitochondria for the treatment of spaceflight-associated mitochondrial damage.

64. Use of autologous or allogeneic mitochondria for the treatment of spaceflight-associated mitochondrial damage, wherein the autologous or allogeneic mitochondria is administered to a subject suffering from spaceflight-associated mitochondrial damage.

65. The use of claim 64, wherein the subject has experienced a spaceflight.

66. The use of claim 65, wherein the spaceflight is at least a week.

67. The use of claim 64, wherein the subject is engaging in spaceflight.

68. The use of claim 64, wherein the subject has been subjected to at least one or more of the following conditions: stress, low gravity conditions, cosmic rays, or radiation.

69. The use of claim 64, wherein the spaceflight-associated mitochondrial damage causes loss of mitochondria in the subject.

70. The use of claim 64, wherein the spaceflight-associated mitochondrial damage comprises oxidative stress and/or inflammation.

71. Use of autologous or allogeneic mitochondria for the treatment of spaceflight-associated mitochondrial damage, wherein the autologous or allogeneic mitochondria is administered to a subject prior to engaging in a spaceflight.

72. The use of claim 71, wherein the subject will experience a long-term spaceflight of at least a week.

73. The use of claim 71, wherein the subject will be subjected to at least one or more of the following conditions: stress, low gravity conditions, cosmic rays, or radiation.

74. The use of claim 71, wherein the spaceflight-associated mitochondrial damage causes loss of mitochondria in the subject.

75. The use of claim 71, wherein the spaceflight-associated mitochondrial damage comprises oxidative stress and/or inflammation.
